(19) United States
(12) Patent Application Publication
(10) Pub. No.: US 2020/0152338 A1
(43) Pub. Date: May 14, 2020

(54) DYNAMICALLY OPTIMIZED INQUIRY PROCESS FOR INTELLIGENT HEALTH PRE-DIAGNOSIS

(71) Applicant: INTERNATIONAL BUSINESS MACHINES CORPORATION, Armonk, NY (US)

(72) Inventors: Xin Zhou, Beijing (CN); Qi Cheng Li, Beijing (CN); Li Jun Mei, Beijing (CN); Hao Chen, Beijing (CN); Ya Bin Dang, Beijing (CN)

(21) Appl. No.: 16/190,691

(22) Filed: Nov. 14, 2018

(51) Int. Cl. G16H 80/00; G16H 50/30; G10L 15/22; G10L 25/66; G10L 13/04

(52) U.S. Cl. CPC G16H 80/00; G16H 50/30; G10L 13/043; G10L 25/66; G10L 15/22

(57) ABSTRACT

A system is provided for facilitating medical conversation. The system includes a user interface, having a Natural Language Processing (NLP) system and an Automatic Speech Recognition (ASR) system, for processing user utterances to extract symptoms, attribute types and attribute values from a user. The system further includes a memory for storing program code. The system also includes a processor for running the program code to transform the symptoms, the attribute types, and the attribute values into a graph and extract relative entities and relationships of the relative entities from the graph. The processor further runs the program code to calculate an Inquiry Efficiency Index (IEI) of each candidate inquiry path based on the relative entities and the relationships of the relative entities. The processor additionally runs the program code to calculate a recommended inquiry path from among the candidate inquiry paths based on the IEI of each candidate inquiry path.

### BACKGROUND

### Technical Field

The present invention generally relates to health diagnosis, and more particularly to a dynamically optimized inquiry process for intelligent health pre-diagnosis.

### Description of the Related Art

Intelligent health pre-diagnosis systems are becoming increasingly popular. Such systems are typically configured to solve medical demand-supply issues, save a user's time cost and provide convenience, and pre-collect symptoms to improve the doctor's effectiveness and efficiency.

However, conventional intelligent health pre-diagnosis systems suffer from various deficiencies. For example, such conventional systems cannot guarantee that necessary information is collected from the user. Moreover, such conventional systems are not designed to reduce the number of conversation turns for symptom collection and clarification. Also, such conventional systems are not designed to reduce the complexity for the user to answer an inquiry. Accordingly, there is a need for an optimized inquiry process for intelligent health pre-diagnosis.

### SUMMARY

According to an aspect of the present invention, a computer processing system is provided for facilitating medical conversation. The computer processing system includes a user interface, having a Natural Language Processing (NLP) system and an Automatic Speech Recognition (ASR) system, for processing user utterances to extract symptoms, attribute types and attribute values from a user. The computer processing system further includes a memory for storing program code. The computer processing system also includes a processor device for running the program code to transform the symptoms, the attribute types, and the attribute values into a graph and extract relative entities and relationships of the relative entities from the graph. The processor device further runs the program code to calculate an Inquiry Efficiency Index (ID) of each of candidate inquiry paths based on the relative entities and the relationships of the relative entities. The processor device additionally runs the program code to calculate a recommended inquiry path from among the candidate inquiry paths based on the IEI of each of the candidate inquiry paths.

According to another aspect of the present invention, a computer-implemented method is provided for facilitating medical conversation. The method includes processing, by a user interface, having a Natural Language Processing (NLP) system and an Automatic Speech Recognition (ASR) system, user utterances to extract symptoms, attribute types and attribute values from a user. The method further includes transforming, by a processor device, the symptoms, the attribute types, and the attribute values into a graph and extracting, by the processor device, relative entities and relationships of the relative entities from the graph. The method also includes calculating, by the processor device, an Inquiry Efficiency Index (ID) of each of candidate inquiry paths based on the relative entities and the relationships of the relative entities. The method additionally includes calculating, by the processor device, a recommended inquiry path from among the candidate inquiry paths based on the IEI of each of the candidate inquiry paths.

According to yet another aspect of the present invention, a computer program product is provided for facilitating medical conversation. The computer program product includes a non-transitory computer readable storage medium having program instructions embodied therewith. The program instructions are executable by a computer to cause the computer to perform a method. The method includes processing, by a user interface of the computer, having a Natural Language Processing (NLP) system and an Automatic Speech Recognition (ASR) system, user utterances to extract symptoms, attribute types and attribute values from a user. The method further includes transforming, by a processor device of the computer, the symptoms, the attribute types, and the attribute values into a graph and extracting, by the processor device, relative entities and relationships of the relative entities from the graph. The method also includes calculating, by the processor device, an Inquiry Efficiency Index (IEI) of each of candidate inquiry paths based on the relative entities and the relationships of the relative entities. The method additionally includes calculating, by the processor device, a recommended inquiry path from among the candidate inquiry paths based on the IEI of each of the candidate inquiry paths.

These and other features and advantages will become apparent from the following detailed description of illustrative embodiments thereof, which is to be read in connection with the accompanying drawings.

### BRIEF DESCRIPTION OF THE DRAWINGS

The following description will provide details of preferred embodiments with reference to the following figures wherein:

FIG. 1 is a block diagram showing an exemplary processing system to which the present invention may be applied, in accordance with an embodiment of the present invention;

FIG. 2 is a block diagram showing an exemplary pre-diagnosis inquiry system, in accordance with an embodiment of the present invention;

FIG. 3 is a block diagram showing an exemplary environment to which the present invention can be applied, in accordance with an embodiment of the present invention;

FIG. 4 is a diagram showing an exemplary inquiry reference model schema, in accordance with an embodiment of the present invention;

FIG. 5 is a diagram showing an exemplary inquiry reference model instance, in accordance with an embodiment of the present invention;

FIG. 6 is a flow diagram showing an exemplary method for generating an inquiry path based on an inquiry reference model, in accordance with an embodiment of the present invention;

FIG. 7 is a diagram showing an exemplary sample inquiry process to which the present invention can be applied, in accordance with an embodiment of the present invention;

FIG. 8 is a flow diagram showing an exemplary method for generating candidates for a next round of inquiry, in accordance with an embodiment of the present invention;

FIG. 9 is a diagram showing an exemplary sample input and an exemplary sample output relating to the method of FIG. 8, in accordance with an embodiment of the present invention;

FIG. 10 is a diagram showing an exemplary path, in accordance with an embodiment of the present invention;

FIG. 11 is a diagram showing an exemplary policy, in accordance with an embodiment of the present invention;

FIG. 12 is a block diagram showing an illustrative cloud computing environment having one or more cloud computing nodes with which local computing devices used by cloud consumers communicate, in accordance with an embodiment of the present invention; and

FIG. 13 is a block diagram showing a set of functional abstraction layers provided by a cloud computing environment, in accordance with an embodiment of the present invention.

### DETAILED DESCRIPTION

The present invention is directed to a dynamically optimized inquiry process for intelligent health pre-diagnosis.

In an embodiment, the present invention can generate an inquiry reference model that describes the symptoms, attributes, their indication to diseases, and their inter-relationships.

In an embodiment, the present invention can quantify the efficiency of an inquiry process from the following two perspectives: (1) minimized conversation turns; and maximized opportunity to identify diseases and especially severe diseases.

In an embodiment, the present invention can dynamically generate/adjust an inquiry path based on a reference model toward the optimal efficiency. In this way, the complexity implicated for a user to answer an inquiry is reduced based on the selected inquiry recommended for the user.

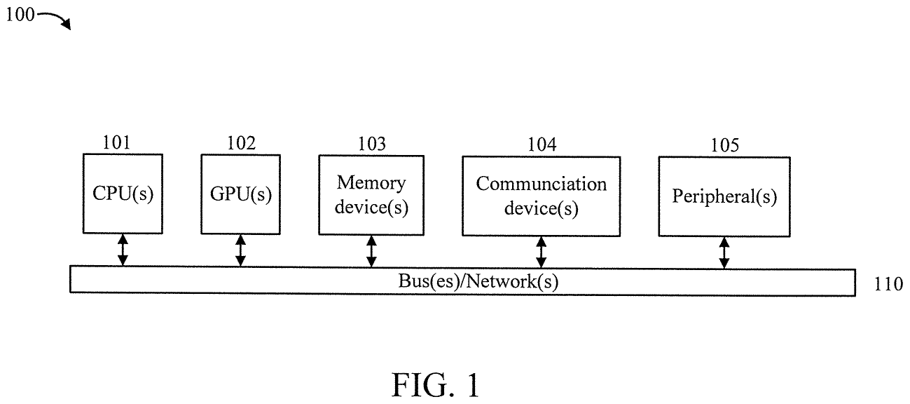

FIG. 1 is a block diagram showing an exemplary processing system 100 to which the present invention may be applied, in accordance with an embodiment of the present invention. The processing system 100 includes a set of processing units (e.g., CPUs) 101, a set of GPUs 102, a set of memory devices 103, a set of communication devices 104, and set of peripherals 105. The CPUs 101 can be single or multi-core CPUs. The GPUs 102 can be single or multi-core GPUs. The one or more memory devices 103 can include caches, RAMs, ROMs, and other memories (flash, optical, magnetic, etc.). The communication devices 104 can include wireless and/or wired communication devices (e.g., network (e.g., WIFI, etc.) adapters, etc.). The peripherals 105 can include a display device, a user input device, a printer, an imaging device, and so forth. Elements of processing system 100 are connected by one or more buses or networks (collectively denoted by the figure reference numeral 110).

In an embodiment, the memory device 103 can be configured, with other elements such as a processor (101 and/or 102), a microphone and speaker (peripherals 105), to implement a Text-To-Speech (TTS) system, an Automatic Speech Recognition (ASR) system, and a Natural Language Processing (NLP) system. Such (TTS, ASR, and NLP) systems can be part of an optimized user interface 199 provided by the present invention in order to dynamically optimize an inquiry process for intelligent health pre-diagnosis. In this way, a conversational dialog can be had with a given patient.

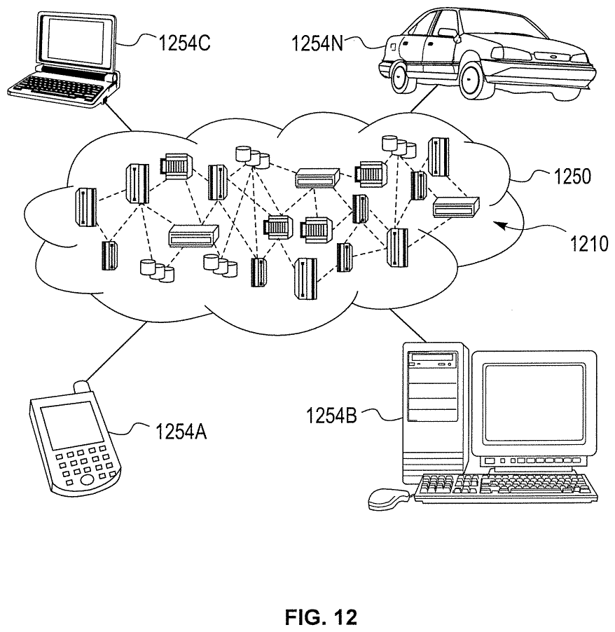

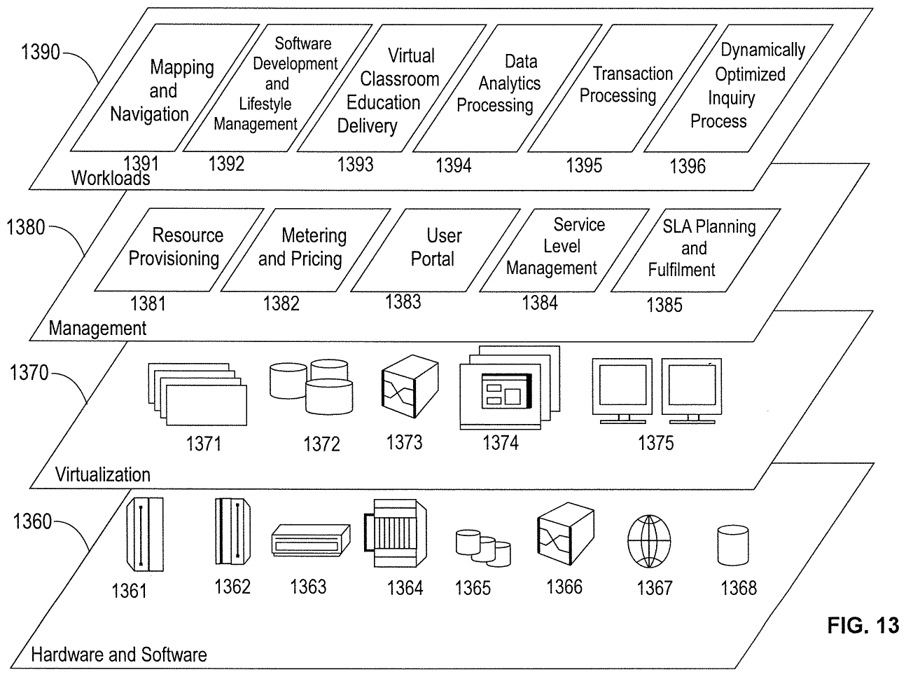

Of course, the processing system 100 may also include other elements (not shown), as readily contemplated by one of skill in the art, as well as omit certain elements. For example, various other input devices and/or output devices can be included in processing system 100, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. Further, in another embodiment, a cloud configuration can be used (e.g., see FIGS. 12-13). These and other variations of the processing system 100 are readily contemplated by one of ordinary skill in the art given the teachings of the present invention provided herein.

Moreover, it is to be appreciated that various figures as described below with respect to various elements and steps relating to the present invention that may be implemented, in whole or in part, by one or more of the elements of system 100.

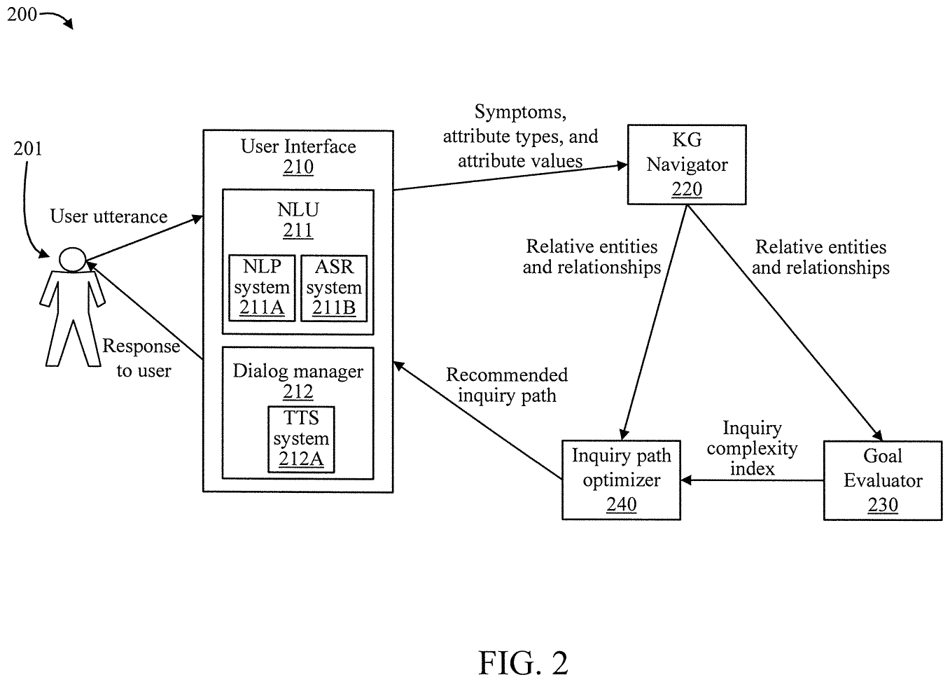

FIG. 2 is a block diagram showing an exemplary pre-diagnosis inquiry system 200, in accordance with an embodiment of the present invention.

The pre-diagnosis inquiry system 200 includes a user interface 210, a KG navigator 220, an inquiry efficiency evaluator (also known as “goal evaluator”) 230, and an inquiry path optimizer 240. It is to be appreciated that some of the preceding components will necessarily involve a processor device and memory.

In an embodiment, the user interface 210 includes a Natural Language Unit (NLU) 211 and a dialog manager 212. The NLU 211 includes a Natural Language Processing (NLP) system 211A and an Automatic Speech Recognition System 211B. The dialog manager 212 includes a Text-To-Speech (TTS) system 212A.

The NLU 221 receives user utterances from a user 201 as inputs and processes the same to output symptoms, attribute types, and attribute values to the KG navigator 220.

The KG navigator 220 receives the symptoms, attribute types, and attribute values from the NLU 221 and processes the same to output relative entities and relationships to both the inquiry efficiency evaluator 230 and the inquiry path optimizer 240. In an embodiment, the relative entities and relationships are output in the form of a graph that models the relationships among the symptoms, attribute types, and attribute values. To that end, the KG navigator traverses the multiple paths of the graph in order to determine the relative entities and relationships. The KG navigator also managers the inquiry reference model (graph) and updates the graph as applicable, depending upon the implementation.

The inquiry efficiency evaluator 230 receives the relative entities and relationships from the KG navigator 220 and processes the same to output an inquiry efficiency index to the inquiry path optimizer 240. The inquiry efficiency index can include the calculations of an Inquiry Efficiency Index (IEI) for path and an IEI for policy, as described in further detail hereinbelow.

The inquiry path optimizer 240 receives the relative entities and relationships and the inquiry efficiency index as inputs and processes the same to output a recommended inquiry path to the dialog manager 222 of the user interface 210. In an embodiment, the inquiry path optimizer 240 identifies multiple potential candidates for following rounds of inquiry content, and uses the inquiry efficiency index to select the optimal potential candidate as a recommended inquiry path.

The dialog manager 220 provides the response to the user. In doing so, the dialog manager can use a Text-To-Speech (TTS) system in order to transform textual and/or other representations of a response into a machine-generated acoustic utterance for the user to hear. In this way, a conversational dialog can be had with the user.

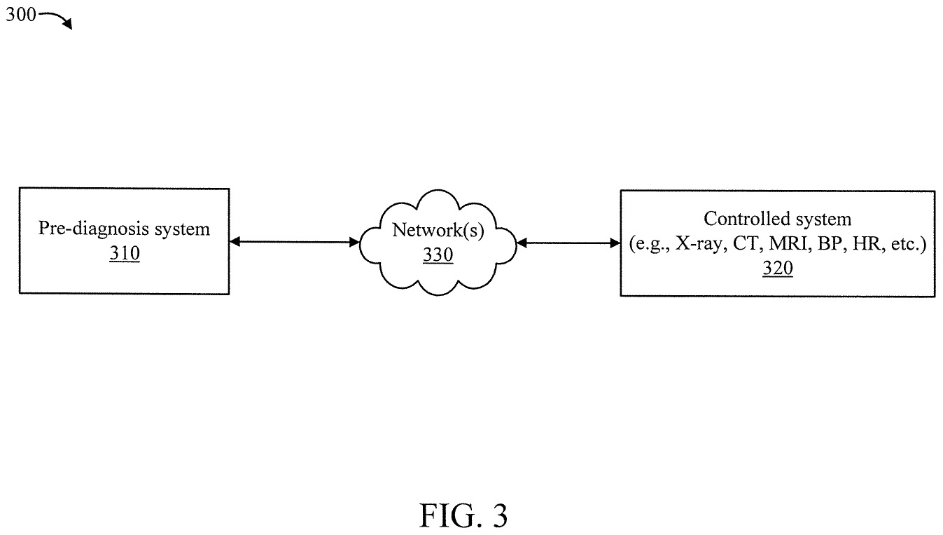

FIG. 3 is a block diagram showing an exemplary environment 300 to which the present invention can be applied, in accordance with an embodiment of the present invention.

The environment 300 includes an pre-diagnosis inquiry system 310 and a controlled system 320. In an embodiment, system 310 is implemented by the pre-diagnosis system 200 of FIG. 2. In an embodiment, system 310 is configured to perform a health pre-diagnosis and can provide action initiation signals to the controlled system depending upon a result of the heath pre-diagnosis.

The pre-diagnosis inquiry system 310 and the controlled system 320 are configured to enable communications therebetween. For example, transceivers and/or other types of communication devices including wireless, wired, and combinations thereof can be used. In an embodiment, communication between the pre-diagnosis inquiry system 310 and the controlled system 320 can be performed over one or more networks, collectively denoted by the figure reference numeral 330. The communication can include, but is not limited to, inspection images (and possible template images as well) from the controlled system 320, and defect detection results and action initiation control signals from the pre-diagnosis inquiry system 310. The controlled system 320 can be any type of processor-based system such as, for example, but not limited to, an imaging machine (e.g., X-ray machine, Computed Tomography (CT) scan machine, Magnetic Resonance Imaging (MRI) machine, etc.), an automatic Blood Pressure machine, a Heart Rate (HR) machine (for measuring, e.g., Beats Per Minute (BPM)), and so forth.

The controlled system 320 provides images to the pre-diagnosis inquiry system 310 which can use the images to determinations regarding defects and perform certain actions in response thereto.

The controlled system 320 can be controlled based on a result (pre-diagnosis) from the pre-diagnosis inquiry system 310. For example, based on a pre-diagnosis, the pre-diagnosis inquiry system 310 can send a control signal to the controlled system 320 to command the controlled system 320 to perform one or more actions. The actions are dependent upon the pre-diagnosis and the implementation. For example, in an embodiment, the controlled system 320 is an imaging machine, and patient specific information obtained from the pre-diagnosis inquiry system 310 can be sent to the controlled system along with one or more commands to perform imaging on a patient with the imaging tailored to the particular patient via the patient specific information. For example, based on the patient specific information, the imaging portion of the machine may be optimally located relevant to a patient's pre-diagnosed condition. In another embodiment, the pre-diagnosis inquiry system 310 can send a command to the controlled system 320, implemented as an automatic blood pressure machine, in order to automatically take a patient's blood pressure. The obtaining of other patient measurements such as temperature, and so forth can be automated and initiated by commands from the pre-diagnosis inquiry system 310 to the controlled system 320. It is to be appreciated that the preceding actions are merely illustrative and, thus, other actions can also be performed, as readily appreciated by one of ordinary skill in the art given the teachings of the present invention provided herein, while maintaining the spirit of the present invention.

In an embodiment, the pre-diagnosis inquiry system 310 can be implemented as a node in a cloud-computing arrangement. In an embodiment, a single pre-diagnosis inquiry system 310 can be assigned to a single controlled system or to multiple controlled systems e.g., different machines in an assembly line of patient measurement machines that are selectively used for a given patient, and so forth). These and other configurations of the elements of environment 300 are readily determined by one of ordinary skill in the art given the teachings of the present invention provided herein, while maintaining the spirit of the present invention.

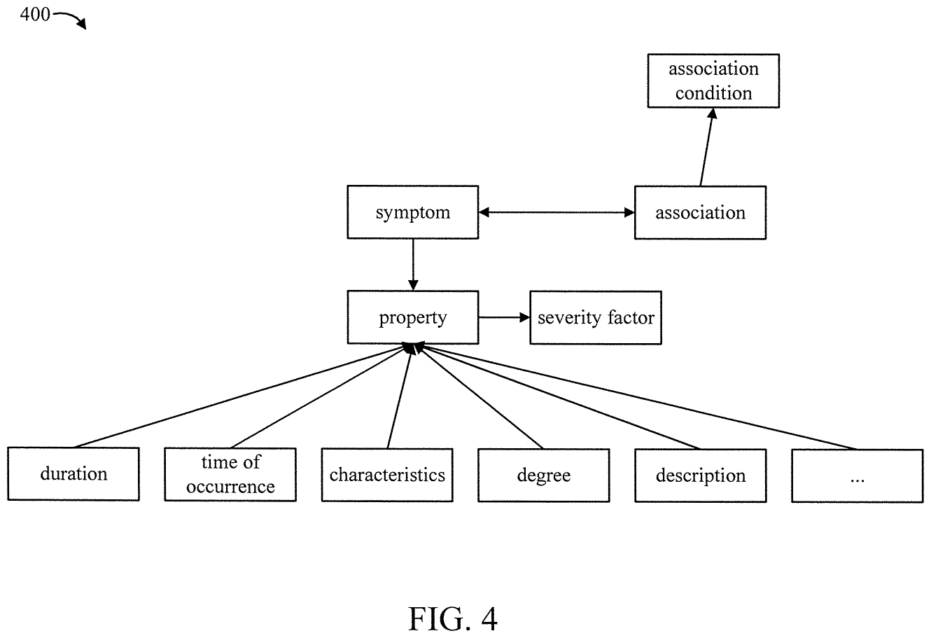

FIG. 4 is a diagram showing an exemplary inquiry reference model schema 400, in accordance with an embodiment of the present invention.

The schema 400 involves the following: duration; time of occurrence; characteristics; degree; description, other inputs indicated by the characters “. . . ”; symptom; association; association condition; and severity factor.

The severity factor relates to the following. Consider a scenario where a symptom or symptom property is associated to N diseases, among which N1 are severe diseases. Then the severity factor of this symptom or symptom property is equal to N1/N.

The flow of information in the schema 400 is indicated as shown by the arrows in FIG. 4.

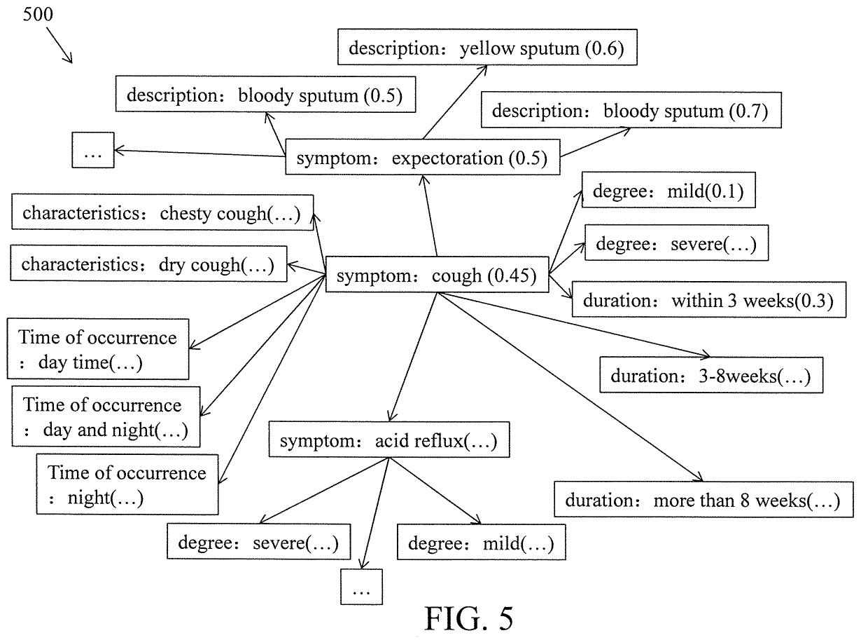

FIG. 5 is a diagram showing an exemplary inquiry reference model instance 500, in accordance with an embodiment of the present invention. In the example of FIG. 5, the inquiry reference model instance 500 relates to various symptoms including: cough; acid reflux; expectoration. Each of the symptoms has various attributes associated therewith and can also be associated with one or more other symptoms. Moreover, each of the symptoms and each of the corresponding attributes have various efficiency values associated therewith. Accordingly, some of the attributes may repeat for a given symptom, but may be associated with different respective efficiency values.

For example, the symptom “cough” has the following other symptoms associated therewith: expectoration; and acid reflux.

The symptom “cough” directly has the following attributes associated therewith: degree, mild; degree, severe; duration, within 3 weeks; duration, 3-8 weeks; duration, more than 8 weeks; time of occurrence, day time; time of occurrence, day and night; time of occurrence, night; characteristics, chesty cough; characteristics, dry cough.

The symptom “cough” also indirectly has the attributes associated with the other symptoms as specified below.

For example, the symptom “expectoration” has the following attributes associated therewith: bloody sputum; yellow sputum; and other attributes indicated by the characters “. . . ”.

The symptom acid reflux has the following attributes associated therewith: degree, severe; degree, mild; and other attributes indicated by the characters “. . . ”.

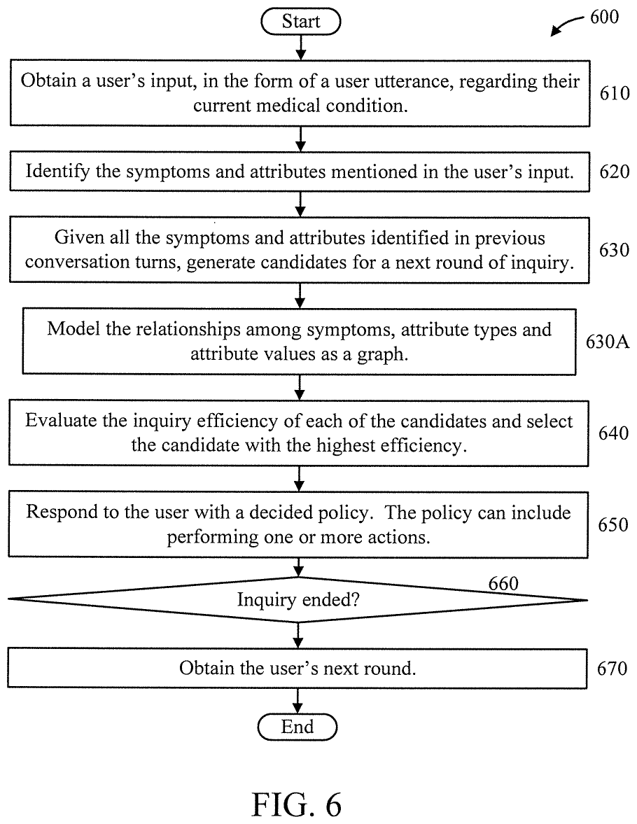

FIG. 6 is a flow diagram showing an exemplary method 600 for generating an inquiry path based on an inquiry reference model, in accordance with an embodiment of the present invention.

At block 610, obtain a user's input, in the form of a user utterance, regarding their current medical condition.

At block 620, identify the symptoms and attributes mentioned in the user's input.

At block 630, given all the symptoms and attributes identified in previous conversation turns, generate candidates for a next round of inquiry.

In an embodiment, block 630 can include block 630A.

At block 630A, model the relationships among symptoms, attribute types and attribute values as a graph.

At block 640, evaluate the inquiry efficiency of each of the candidates and select the candidate with the highest efficiency.

At block 650, respond to the user with a decided policy. The policy can include performing one or more actions. For example, exemplary actions are described with respect to the controlled system 320 of FIG. 3.

At block 660, determine whether or not the inquiry has ended. If so, then terminate the method. Otherwise, proceed to step 670.

At step 670, obtain the user's next round.

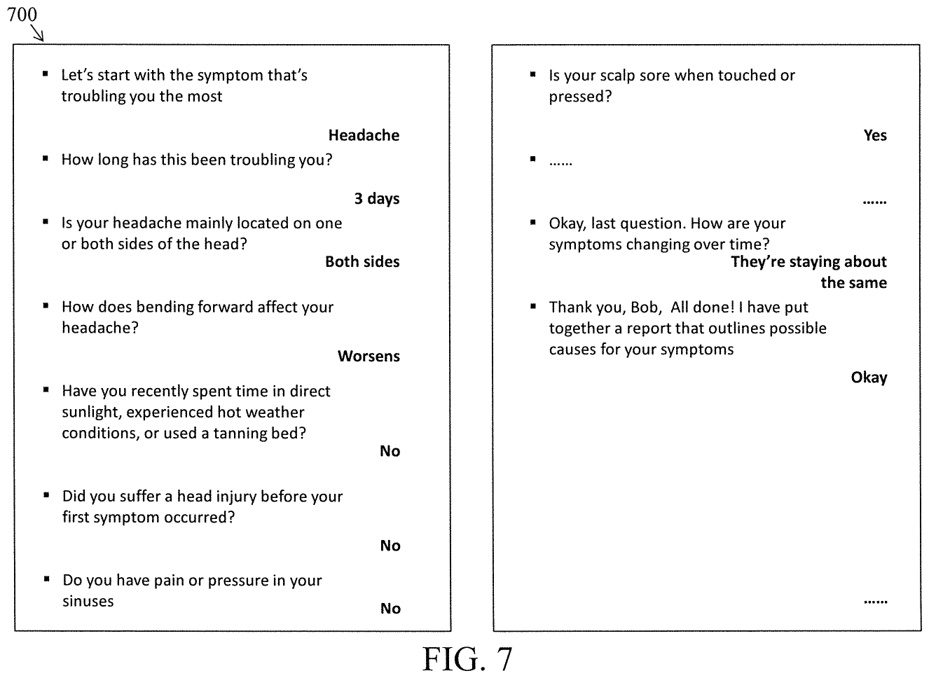

FIG. 7 is a diagram showing an exemplary sample inquiry process 700 to which the present invention can be applied, in accordance with an embodiment of the present invention. The sample inquiry process 700 is shown relative to two panels, where in each panel the utterances for one speaker are on the left side in non-bolded font, and the utterances for another speaker are on the right side in bolded font.

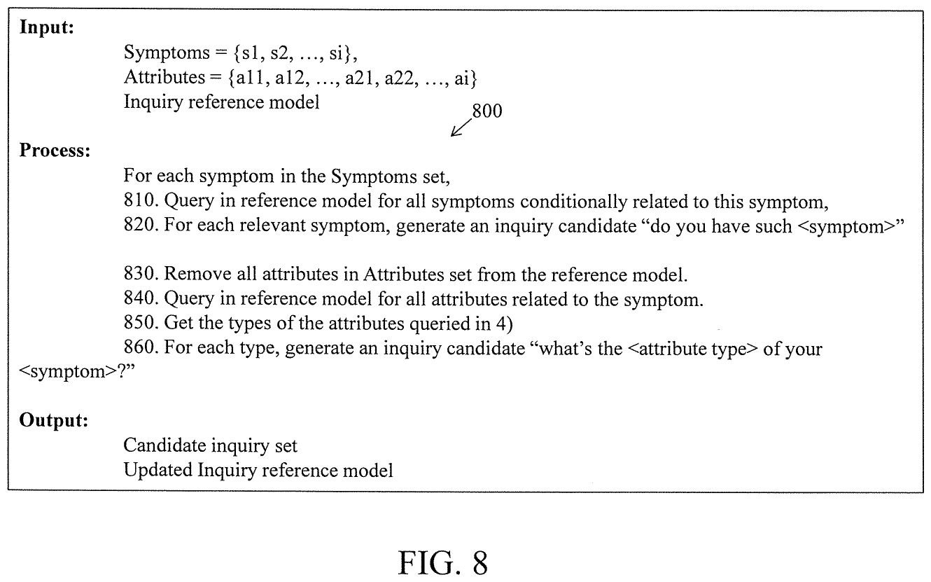

FIG. 8 is a flow diagram showing an exemplary method 800 for generating candidates for a next round of inquiry, in accordance with an embodiment of the present invention.

The input to method 800 can include the following:
(1) Symptoms={s1, s2, . . . , si};
(2) Attributes={a11, a12, . . . , a21, a22, . . . , ai}; and
(3) Inquiry reference model.

The output from method 800 can include the following:
(1) Candidate inquiry set; and
(2) Updated inquiry reference model.

At block 810, for each symptom in the symptoms set, query in the reference model for all symptoms conditionally related to this symptom.

At block 820, for each relevant symptom, generate an inquiry candidate “do you have such <symptom>?”.

At block 830, remove all attributes in the attributes set from the reference model.

At block 840, query in the reference model for all attributes related to the symptom.

At block 850, obtain the types of the attributes queried in block 840.

At block 860, for each type, generate an inquiry candidate “what's the <attribute type> of your <symptom>?”

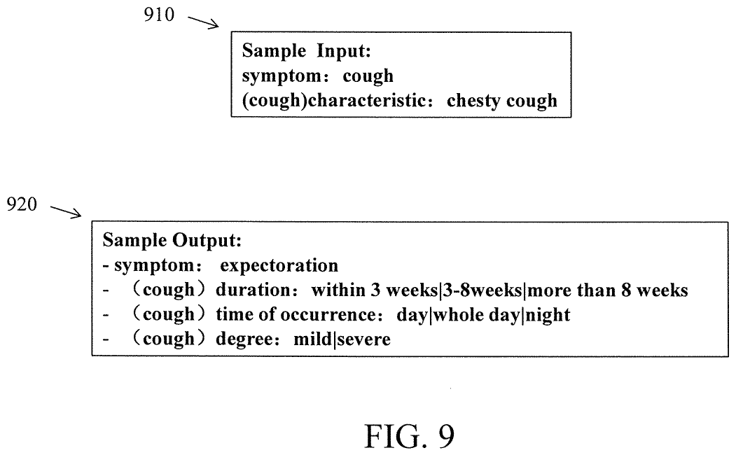

FIG. 9 is a diagram showing an exemplary sample input 910 and an exemplary sample output 920 relating to the method 800 of FIG. 8, in accordance with an embodiment of the present invention.

The sample input 910 involves the symptom cough, and the (cough) characteristic of a chesty cough.

The sample output involves the symptom expectoration, the (cough) duration of any of (within 3 weeks, 3-8 weeks, and more than 8 weeks), the (cough) time of occurrence of any of (day, whole day, and night), and the (cough) degree of any of mild and severe.

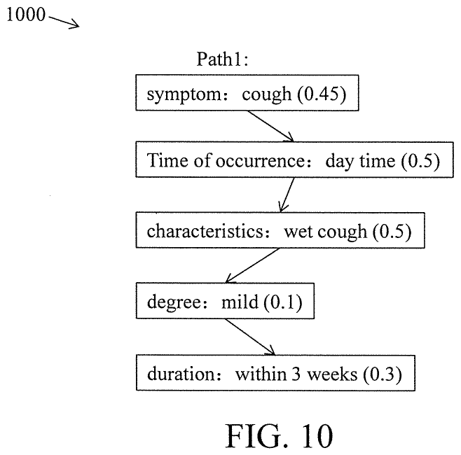

FIG. 10 is a diagram showing an exemplary “path 1” 1000, in accordance with an embodiment of the present invention.

Path 1 1000 involves the following: symptom, cough; time of occurrence, day time; characteristics, wet cough; degree, mild; and duration, within 3 weeks.

An Inquiry Efficiency Index (IEI) for path is calculated as follows:

$$IEI ( path ) = {\sum }_{i = 1}^{n} ( \frac{si}{i} )$$

where sᵢ denotes a severity factor of the symptom.

Hence, for path 1, the following calculation applies:

IEI(path1)=0.45/1+0.5/2+0.5/3+0.1/4+0.3/5=0.955

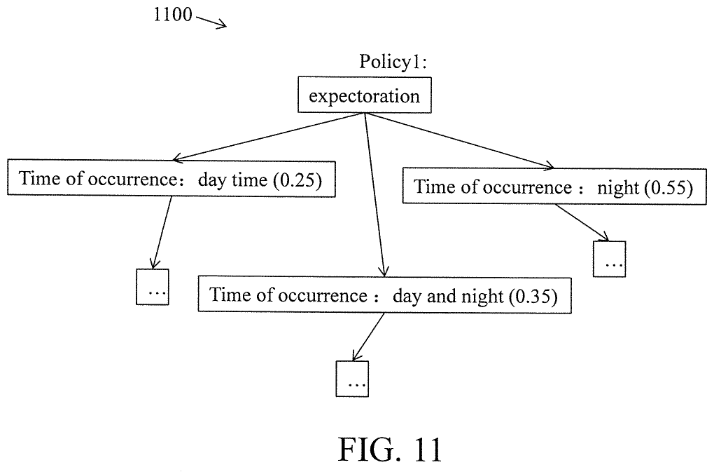

FIG. 11 is a diagram showing an exemplary “policy 1” 1100, in accordance with an embodiment of the present invention.

The policy 1 1100 involves the following: expectoration; time of occurrence, day time; time of occurrence, night; time of occurrence, day and night.

An Inquiry Efficiency Index (IEI) for policy is calculated as follows:

$$IEI ( policyi , i ) = \frac{s}{i} + {\sum }_{j = 1}^{m} ( IEI ( policy , i + 1 ) ) \frac{1}{m}$$

where s denotes the severity factor of the root symptom/symptom property of policy i, and m denotes the total number of sub nodes of the root symptom/symptom property.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

### Characteristics are as Follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

### Service Models are as Follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

### Deployment Models are as Follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

Referring now to FIG. 12, illustrative cloud computing environment 1250 is depicted. As shown, cloud computing environment 1250 includes one or more cloud computing nodes 1210 with which local computing devices used by cloud consumers, such as, for example, personal digital assistant (PDA) or cellular telephone 1254A, desktop computer 1254B, laptop computer 1254C, and/or automobile computer system 1254N may communicate. Nodes 1210 may communicate with one another. They may be grouped (not shown) physically or virtually, in one or more networks, such as Private, Community, Public, or Hybrid clouds as described hereinabove, or a combination thereof. This allows cloud computing environment 1250 to offer infrastructure, platforms and/or software as services for which a cloud consumer does not need to maintain resources on a local computing device. It is understood that the types of computing devices 1254A-N shown in FIG. 12 are intended to be illustrative only and that computing nodes 1210 and cloud computing environment 1250 can communicate with any type of computerized device over any type of network and/or network addressable connection (e.g., using a web browser).

Referring now to FIG. 13, a set of functional abstraction layers provided by cloud computing environment 1250 (FIG. 12) is shown. It should be understood in advance that the components, layers, and functions shown in FIG. 13 are intended to be illustrative only and embodiments of the invention are not limited thereto. As depicted, the following layers and corresponding functions are provided:

Hardware and software layer 1360 includes hardware and software components. Examples of hardware components include: mainframes 1361; RISC (Reduced Instruction Set Computer) architecture based servers 1362; servers 1363; blade servers 1364; storage devices 1365; and networks and networking components 1366. In some embodiments, software components include network application server software 1367 and database software 1368.

Virtualization layer 1370 provides an abstraction layer from which the following examples of virtual entities may be provided: virtual servers 1371; virtual storage 1372; virtual networks 1373, including virtual private networks; virtual applications and operating systems 1374; and virtual clients 1375.

In one example, management layer 1380 may provide the functions described below. Resource provisioning 1381 provides dynamic procurement of computing resources and other resources that are utilized to perform tasks within the cloud computing environment. Metering and Pricing 1382 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 1383 provides access to the cloud computing environment for consumers and system administrators. Service level management 1384 provides cloud computing resource allocation and management such that required service levels are met. Service Level Agreement (SLA) planning and fulfillment 1385 provide pre-arrangement for, and procurement of, cloud computing resources for which a future requirement is anticipated in accordance with an SLA.

Workloads layer 1390 provides examples of functionality for which the cloud computing environment may be utilized. Examples of workloads and functions which may be provided from this layer include: mapping and navigation 1391; software development and lifecycle management 1392; virtual classroom education delivery 1393; data analytics processing 1394; transaction processing 1395; and dynamically optimized inquiry process for intelligent health pre-diagnosis 1396.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as SMALLTALK, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

Having described preferred embodiments of a system and method (which are intended to be illustrative and not limiting), it is noted that modifications and variations can be made by persons skilled in the art in light of the above teachings. It is therefore to be understood that changes may be made in the particular embodiments disclosed which are within the scope of the invention as outlined by the appended claims. Having thus described aspects of the invention, with the details and particularity required by the patent laws, what is claimed and desired protected by Letters Patent is set forth in the appended claims.

What is claimed is:

1. A computer processing system for facilitating medical conversation, comprising:
a user interface, having a Natural Language Processing (NLP) system and an Automatic Speech Recognition (ASR) system, for processing user utterances to extract symptoms, attribute types and attribute values from a user;
a memory for storing program code; and
a processor device for running the program code to
transform the symptoms, the attribute types, and the attribute values into a graph and extract relative entities and relationships of the relative entities from the graph;
calculate an Inquiry Efficiency Index (IEI) of each of candidate inquiry paths based on the relative entities and the relationships of the relative entities; and
calculate a recommended inquiry path from among the candidate inquiry paths based on the IEI of each of the candidate inquiry paths.

2. The computer processing system of claim 1, wherein the IEI is calculated recursively.

3. The computer processing system of claim 1, wherein the IEI is calculated to quantify an inquiry process efficiency relative to minimizing conversation turns, and maximizing a disease diagnosis.

4. The computer processing system of claim 1, wherein the recommended inquiry path is calculated with a bias towards an optimal efficiency.

5. The computer processing system of claim 1, wherein the user interface is configured to receive user updates to the inquiry reference model.

6. The computer processing system of claim 1, wherein the processor device further calculates an ID of each of candidate inquiry policies, and wherein the processor device calculates the recommended inquiry path from among the candidate inquiry paths further based on the ID of each of the candidate inquiry policies.

7. The computer processing system of claim 1, wherein the recommended inquiry path comprises a question directed to determining one or more attribute types of a given symptom.

8. The computer processing system of claim 1, wherein the user interface further comprises a Text-To-Speech system for transforming the recommended inquiry path into a representative acoustic utterance.

9. The computer processing system of claim 1, wherein the recommended inquiry path is dynamically adjusted to achieve an optimal efficiency relative to other ones of the candidate inquiry paths.

10. A computer-implemented method for facilitating medical conversation, the method comprising:
processing, by a user interface, having a Natural Language Processing (NLP) system and an Automatic Speech Recognition (ASR) system, user utterances to extract symptoms, attribute types and attribute values from a user;
transforming, by a processor device, the symptoms, the attribute types, and the attribute values into a graph and extracting, by the processor device, relative entities and relationships of the relative entities from the graph;
calculating, by the processor device, an Inquiry Efficiency Index (ID) of each of candidate inquiry paths based on the relative entities and the relationships of the relative entities; and
calculating, by the processor device, a recommended inquiry path from among the candidate inquiry paths based on the IEI of each of the candidate inquiry paths.

11. The computer-implemented method of claim 10, wherein the IEI is calculated recursively.

12. The computer-implemented method of claim 10, wherein the IEI is calculated to quantify an inquiry process efficiency relative to minimizing conversation turns, and maximizing a disease diagnosis.

13. The computer-implemented method of claim 10, wherein the recommended inquiry path is calculated with a bias towards an optimal efficiency.

14. The computer-implemented method of claim 10, further comprising configuring the user interface to receive user updates to the inquiry reference model.

15. The computer-implemented method of claim 10, wherein the processor device further calculates an ID of each of candidate inquiry policies, and wherein the processor device calculates the recommended inquiry path from among the candidate inquiry paths further based on the ID of each of the candidate inquiry policies.

16. The computer-implemented method of claim 10, wherein the recommended inquiry path comprises a question directed to determining one or more attribute types of a given symptom.

17. The computer-implemented method of claim 10, wherein the user interface further comprises a Text-To-Speech system for transforming the recommended inquiry path into a representative acoustic utterance.

18. The computer-implemented method of claim 10, wherein the recommended inquiry path is dynamically adjusted to achieve an optimal efficiency relative to other ones of the candidate inquiry paths.

19. A computer program product for facilitating medical conversation, the computer program product comprising a non-transitory computer readable storage medium having program instructions embodied therewith, the program instructions executable by a computer to cause the computer to perform a method comprising:
processing, by a user interface of the computer, having a Natural Language Processing (NLP) system and an Automatic Speech Recognition (ASR) system, user utterances to extract symptoms, attribute types and attribute values from a user;
transforming, by a processor device of the computer, the symptoms, the attribute types, and the attribute values into a graph and extracting, by the processor device, relative entities and relationships of the relative entities from the graph;
calculating, by the processor device, an Inquiry Efficiency Index (ID) of each of candidate inquiry paths based on the relative entities and the relationships of the relative entities; and
calculating, by the processor device, a recommended inquiry path from among the candidate inquiry paths based on the IEI of each of the candidate inquiry paths.

20. The computer program product of claim 19, wherein the IEI is calculated to quantify an inquiry process efficiency relative to minimizing conversation turns, and maximizing a disease diagnosis.
